SEQUENCE LISTING (12 sequences)

SEQ ID NO: 1 - DNA, 28 bp, Artificial Sequence
  PCR primer
gagacataatggtgatggctaggaccca

SEQ ID NO: 2 - DNA, 28 bp, Artificial Sequence
  PCR primer
ctgcgacagatattccctggaccaatcc

SEQ ID NO: 3 - DNA, 26 bp, Artificial Sequence
  PCR primer
ccatcctaatacgactcactataggc

SEQ ID NO: 4 - DNA, 23 bp, Artificial Sequence
  PCR primer
actcactatagggctcgagcggc

SEQ ID NO: 5 - DNA, 50 bp, Artificial Sequence
  PCR primer
ggcattcgaattcgccgccaccatgaatgagaaatgggacacaaactctt

SEQ ID NO: 6 - DNA, 50 bp, Artificial Sequence
  PCR primer
aggattatcactctagatctttttaaatctcactgctgttagtagtttct

SEQ ID NO: 7 - DNA, 27 bp, Artificial Sequence
  PCR primer
tggcacgtggtgtccaggaagaagcag

SEQ ID NO: 8 - DNA, 1440 bp, Human
ttgagccggcagactgcgaaaagtagctggagccggagcagggacagaacctgttgctgcagacgggcttggtggattctggttcctgccgccgacagggctcgccgggagaggttcatcatgaatgagaaatgggacacaaactcttcagaaaactggcatcccatctggaatgtcaatgacacaaagcatcatctgtactcagatattaatattacctatgtgaactactatcttcaccagcctcaagtggcagcaatcttcattatttcctactttctgatcttctttttgtgcatgatgggaaatactgtggtttgctttattgtaatgaggaacaaacatatgcacacagtcactaatctcttcatcttaaacctggccataagtgatttactagttggcatattctgcatgcctataacactgctggacaatattatagcaggatggccatttggaaacacgatgtgcaagatcagtggattggtccagggaatatctgtcgcagcttcagtctttacgttagttgcaattgctgtagataggttccagtgtgtggtctacccttttaaaccaaagctcactatcaagacagcgtttgtcattattatgatcatctgggtcctagccatcaccattatgtctccatctgcagtaatgttacatgtgcaagaagaaaaatattaccgagtgagactcaactcccagaataaaaccagtccagtctactggtgccgggaagactggccaaatcaggaaatgaggaagatctacaccactgtgctgtttgccaacatctacctggctcccctctccctcattgtcatcatgtatggaaggattggaatttcactcttcagggctgcagttcctcacacaggcaggaagaaccaggagcagtggcacgtggtgtccaggaagaagcagaagatcattaagatgctcctgattgtggccctgctttttattctctcatggctgcccctgtggactctaatgatgctctcagactacgctgacctttctccaaatgaactgcagatcatcaacatctacatctacccttttgcacactggctggcattcggcaacagcagtgtcaatcccatcatttatggtttcttcaacgagaatttccgccgtggtttccaagaagctttccagctccagctctgccaaaaaagagcaaagcctatggaagcttataccctaaaagctaaaagccatgtgctcataaacacatctaatcagcttgtccaggaatctacatttcaaaaccctcatggggaaaccttgctttataggaaaagtgctgaaaaaccccaacaggaattagtgatggaagaattaaaagaaactactaacagcagtgagatttaaaaagagctagtgtgataatcctaactctactacgcattatatatttaaatccattgc

SEQ ID NO: 9 - protein, 420 aa, Human
MNEKWDTNSSENWHPIWNVNDTKHHLYSDINITYVNYYLHQPQVAAIFIISYFLIFFLCMMGNTVVCFIVMRNKHMHTVTNLFILNLAISDLLVGIFCMPITLLDNIIAGWPFGNTMCKISGLVQGISVAASVFTLVAIAVDRFQCVVYPFKPKLTIKTAFVIIMIIWVLAITIMSPSAVMLHVQEEKYYRVRLNSQNKTSPVYWCREDWPNQEMRKIYTTVLFANIYLAPLSLIVIMYGRIGISLFRAAVPHTGRKNQEQWHVVSRKKQKIIKMLLIVALLFILSWLPLWTLMMLSDYADLSPNELQIINIYIYPFAHWLAFGNSSVNPIIYGFFNENFRRGFQEAFQLQLCQKRAKPMEAYTLKAKSHVLINTSNQLVQESTFQNPHGETLLYRKSAEKPQQELVMEELKETTNSSEI

SEQ ID NO: 10 - protein, 384 aa, Human
MNSTLFSQVENHSVHSNFSEKNAQLLAFENDDCHLPLAMIFTLALAYGAVIILGVSGNLALIIIILKQKEMRNVTNILIVNLSFSDLLVAIMCLPFTFVYTLMDHWVFGEAMCKLNPFVQCVSITVSIFSLVLIAVERHQLIINPRGWRPNNRHAYVGIAVIWVLAVASSLPFLIYQVMTDEPFQNVTLDAYKDKYVCFDQFPSDSHRLSYTTLLLVLQYFGPLCFIFICYFKIYIRLKRRNNMMDKMRDNKYRSSETKRINIMLLSIVVAFAVCWLPLTIFNTVFDWNHQIIATCNHNLLFLLCHLTAMISTCVNPIFYGFLNKNFQRDLQFFFNFCDFRSRDDDYETIAMSTMHTDVSKTSLKQASPVAFKKINNNDDNEKI

SEQ ID NO: 11 - protein, 444 aa, Human
MSGTKLEDSPPCRNWSSASELNETQEPFLNPTDYDDEEFLRYLWREYLHPKEYEWVLIAGYIIVFVVALIGNVLVCVAVWKNHHMRTVTNYFIVNLSLADVLVTITCLPATLVVDITETWFFGQSLCKVIPYLQTVSVSVSVLTLSCIALDRWYAICHPLMFKSTAKRARNSIVIIWIVSCIIMIPQAIVMECSTVFPGLANKTTLFTVCDERWGGEIYPKMYHICFFLVTYMAPLCLMVLAYLQIFRKLWCRQIPGTSSVVQRKWKPLQPVSQPRGPGQPTKSRMSAVAAEIKQIRARRKTARMLMVVLLVFAICYLPISILNVLKRVFGMFAHTEDRETVYAWFTFSHWLVYANSAANPIIYNFLSGKFREEFKAAFSCCCLGVHHRQEDRLTRGRTSTESRKSLTTQISNFDNISKLSEQVVLTSISTLPAANGAGPLQNW

SEQ ID NO: 12 - protein, 428 aa, Human
MDVVDSLLVNGSNITPPCELGLENETLFCLDQPRPSKEWQPAVQILLYSLIFLLSVLGNTLVITVLIRNKRMRTVTNIFLLSLAVSDLMLCLFCMPFNLIPNLLKDFIFGSAVCKTTTYFMGTSVSVSTFNLVAISLFRYGAICKPLQSRVWQTKSHALKVIAATWCLSFTIMTPYPIYSNLVPFTKNNNQTANMCRFLLPNDVMQQSWHTFLLLILFLIPGIVMMVAYGLISLELYQGIKFEASQKKSAKERKPSTTSSGKYEDSDGCYLQKTRPPRKLELRQLSTGSSSRANRIRSNSSAANLMAKKRVIRMLIVIVVLFFLCWMPIFSANAWRAYDTASAERRLSGTPISFILLLSYTSSCVNPIIYCFMNKRFRLGFMATFPCCPNPGPPGARGEVGEEEEGGTTGASLSRFSYSHMSASVPPQ

(19) United States
(12) Patent Application Publication
(10) Pub. No.: US 2003/0139589 A1
(43) Pub. Date: Jul. 24, 2003

(54) G protein coupled receptor A4

(75) Inventor: Roman Zastawny, Etobicoke (CA)

Correspondence Address: ROTHWELL, FIGG, ERNST & MANBECK, P.C., 1425 K STREET, N.W., SUITE 800, WASHINGTON, DC, 20005, US

(73) Assignee: Allelix Biopharmaceuticals Inc.

(21) Appl. No.: 10/060,369

(22) Filed: Feb. 1, 2002

Related U.S. Application Data
(63) Continuation of application No. 09/173,565, filed on Oct. 16, 1998, now abandoned.

(51) Int. Cl. C12P 21/02; C12N 5/06; C07K 14/705

(52) U.S. Cl. 536/23.5; 530/350; 435/69.1; 435/320.1; 435/325

(57) ABSTRACT

A novel G protein coupled receptor family is described, herein called A4. DNA coding for this receptor has been isolated. Methods of producing recombinant cell lines which produce the receptor as a heterologous membrane-bound product are described, as well as other related aspects of the invention, which are of commercial significance, including use of the cell lines for the discovery of therapeutic compounds which modulate the receptor activity.

### FIELD OF THE INVENTION

This invention relates to the cloning and expression of DNA coding for a novel G protein coupled receptor A4.

### BACKGROUND TO THE INVENTION

G protein coupled receptors have been implicated in many important biological processes in a wide variety of living organisms and include a wide range of biologically active receptors, such as hormone, growth factor and neuroreceptors. For example, adrenergic agents and dopamine (Kobilka et al, PNAS, 84:46-50 (1987); Kobilka et al. Science, 238:650-656 (1987); Bunzow et al, Nature 336:783-787 (1998)); calcitonin; cAMP; adenosine; muscarinic; serotonin all act through G protein coupled receptors.

Members of this class share a common signalling mechanism which involves intracellular transducer elements called G proteins. Briefly, when a chemical messenger binds to the active site of the receptor, the conformation of the receptor changes thereby allowing it to interact with and activate a G protein. The activated G protein causes a molecule of guanosine diphosphate (GDP), that is bound to the surface of the G protein, to be replaced with a molecule of guanosine triphosphate, which causes another alteration in the conformation of the G protein. With GTP bound to its surface the G protein can regulate the activity of an effector. These effectors include enzymes such as adenylyl cyclase and phospholipase C, certain transport proteins and ion channels such as those specific for calcium ions, potassium ions or sodium ions.

G protein coupled receptors have been characterised as having seven putative transmembrane domains each of the order of 20 to 30 hydrophobic amino acids, connecting at least eight divergent hydrophilic loops. The transmembrane regions are designated TM1, TM2 etc. TM3 is implicated in ligand binding signal transduction. Additionally, TM5 and TM6 are implicated in ligand binding. Post translational events such as phosphorylation and lipidation can influence receptor activity.

In view of the diverse functions of G protein coupled receptors, it is not surprising that many therapeutic drugs act by directly modifying the function of G protein coupled receptors.

### SUMMARY OF THE INVENTION

The present invention relates to an isolated polynucleotide encoding a novel mammalian G protein coupled receptor. In one of its aspects the invention thus provides an isolated nucleotide, consisting either of DNA or of RNA, which codes for a G protein coupled receptor or for a fragment or variant thereof.

In another aspect of the present invention, there is provided a cell that has been genetically engineered to produce a G protein coupled receptor herein-defined as an A4 receptor. In related aspects of the present invention, there are provided recombinant DNA constructs and relevant methods useful to create such cells.

In another aspect of the present invention, there is provided a method for evaluating interaction between a test ligand and an A4 receptor, which comprises the steps of incubating the test ligand with a cell that produces an A4 receptor, or with a membrane preparation derived therefrom, and then assessing said interaction by determining at least one of receptor/ligand binding, ligand-induced current, or second messenger response, such as modulation of cAMP or intracellular calcium levels.

Other aspects of the present invention, which encompass various applications of the discoveries herein described, will become apparent from the following detailed description, and from the accompanying drawings.

### BRIEF REFERENCE TO THE FIGURES

FIG. 1 provides a polynucleotide encoding the human A4 receptor and the predicted amino acid sequence.

FIG. 2 provides the percentage similarity and identity between the predicted amino acid sequence of the human A4 receptor and related mammalian receptors.

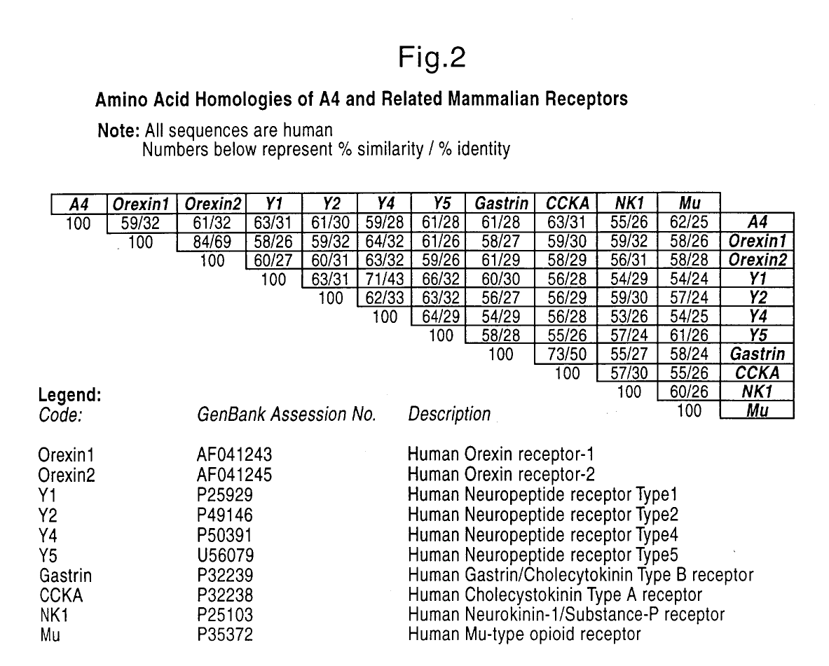

FIG. 3 provides the sequence alignment between the predicted amino acid sequence of the human A4 receptor and the human Y1 receptor.

FIG. 4 provides the sequence alignment between the predicted amino acid sequence of the human A4 receptor and the human Orexin Receptor-2.

FIG. 5 provides the sequence alignment between the predicted amino acid sequence of the human A4 receptor and the human CCK receptor.

FIG. 6 illustrates the FISH mapping results for the A4 receptor/probe 613 on human chromosome 4.

### DETAILED DESCRIPTION OF THE INVENTION AND ITS PREFERRED EMBODIMENTS

The invention relates to G-protein coupled receptors of mammalian origin, including human, and is directed more particularly to a novel G protein coupled receptor, herein designated the A4 receptor, and to isolated polynucleotides encoding these receptors. As used herein “isolated” means separated from polynucleotides that encode other proteins. In the context of polynucleotide libraries, for instance, an A4 receptor-encoding polynucleotide is considered “isolated” when it, or a clone incorporating it, has been selected, and hence removed from association with other polynucleotides within the library. Such polynucleotides may be in the form of RNA, or in the form of DNA, including: cDNA; genomic DNA; and synthetic DNA.

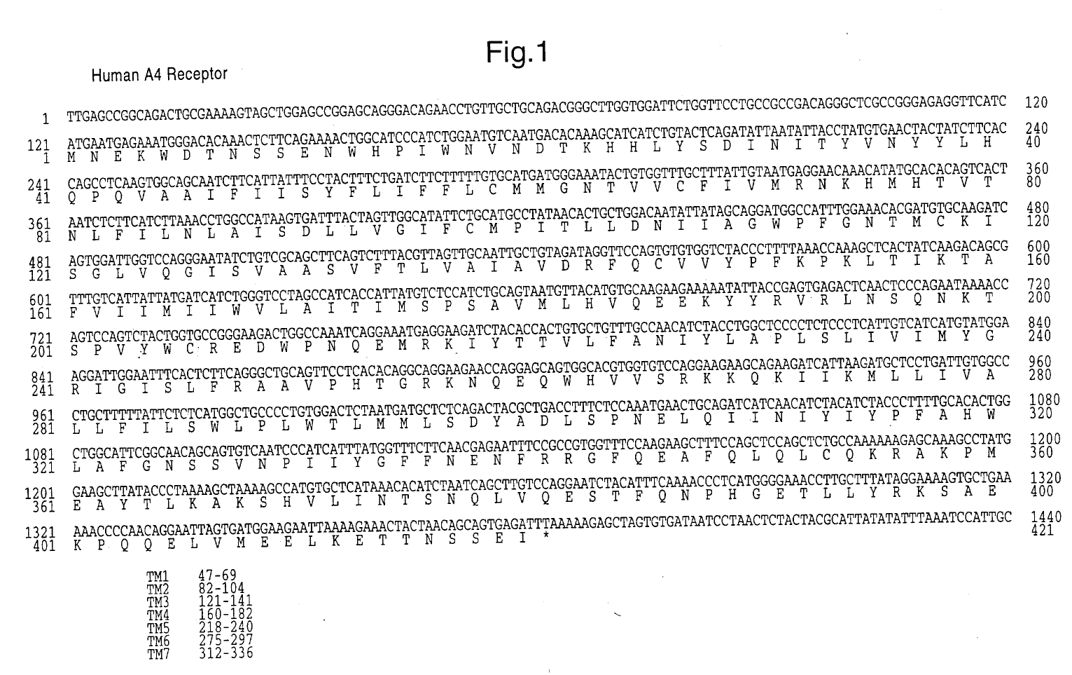

The present invention further relates to variants of the A4 polynucleotide described herein which encode fragments, analogs and derivatives of the peptides having the derived amino acid sequence of FIG. 1. The variants of the polynucleotide may be naturally occurring allelic variants or non-naturally occurring variants of the polynucleotides wherein the synonymous codon is substituted for the native sequence.

As used herein, the term “A4 receptor” is intended to embrace receptors and functional variants that are structurally related thereto, i.e. share at least 46% nucleic acid identity therewith, and more preferably at least 70% nucleic acid identity therewith, including naturally occurring and synthetically derived variants. Naturally occuring variants include mammalian species homologs of the human A4 receptor, in particular the human A4 receptor. Synthetically derived variants of the A4 receptor include ligand binding variants that incorporate one or more, e.g. 1-10, amino acid substitutions, deletions or additions, relative to the human or naturally occuring variants of the human receptor. Generally, it will be desirable that such synthetic variants retain the ligand binding and signal transducing activities of the naturally occurring receptor. Therefore, preferably above-mentioned substitutions, deletions or additions will be conservative in nature i.e. relate to positions in the amino acid sequence wherein such modifications do not result in complete loss of receptor function, that is ligand binding and/or ability to signal transduction. The amino acid sequence of the A4 receptor has greater than 32-40% identity, preferably greater than 55-65% identity, more preferably greater than 70% identity and most preferably greater than 95% identity, to the predicted amino acid sequence of FIG. 1.

As used herein the terms fragment, derivative and analog mean a polypeptide which either retains substantially the same biological function or activity of A4 i.e functions as a G protein coupled receptor, or retains the ability to bind the ligand, for example a soluble form of the receptor. Fragments also include portions of the A4 protein which are useful for raising antibodies, detailed hereinbelow.

Like other members of the G protein coupled receptor family, receptor subtype A4 is characterised by a pharmacological profile i.e. a ligand binding “signature”. Thus, in a key aspect of the present invention, the A4 receptor is exploited for the purpose of screening candidate ligands, including candidate drug compounds, which have the ability to interact with the A4 receptor and/or the ability to compete with endogenous A4 receptor ligands. In one embodiment, candidate ligands to be screened are peptides. In a more preferred embodiment candidate ligands are NPY, peptide YY, orexin, CCK, gastrin, substance P or substance K. Most preferably, candidate ligands are NPY, oxerin, CCK or gastrin and peptide analogs of those.

A polynucleotide encoding a polypeptide of the present invention has been found in adult human kidney, liver, lung and placenta The human polynucleotide is structurally related to the G protein coupled receptor family. It contains an open reading frame encoding a protein of 420 amino acids. The A4 receptor protein exhibits the highest degree of homology to the orexin receptor family with 32% identity and 59-61% similarity over the entire amino acid sequences. A4 also shows significant homology to the NPY, Gastrin and CCKA receptors, among others. These receptors possess structural features characteristic of the G protein coupled receptors in general, including an extracellular N-terminus and an intracellular C-terminus, as well as seven transmembrane domains which serve to anchor the receptor within the cell surface membrane. These receptors are further characterised by their coupling to G-proteins, or guanine nucleotide regulatory proteins. With respect to structural domains of the human A4 receptor, hydropathy analysis reveals seven putative transmembrane domains: one spanning residues 47-69 inclusive (TM-1); another spanning residues 82-104 (TM-2); a third spanning residues 121-141 (TM-3); a fourth spanning residues 160-182 (TM-4); a fifth spanning residues 218-240 (TM-5); a sixth spanning residues 275-297 (TM-6); and a seventh spanning residues 312-336 (TM-7). Based on this assignment, it is likely that the A4 receptor structure, in its natural membrane-bound form, consists of a 46 amino acid N-terminal extracellular domain, followed by a hydrophobic region containing seven transmembrane domains and an intracellular 84 amino acid C-terminal domain.

The invention also relates to polynucleotides which hybridise to the hereinabove described sequences if there is at least 46% and preferably 55% homology between A4and the hybridising sequences. Most preferably, the hybridising sequences show at least 70% homology to the sequences described herein. In particular, the invention relates to polynucleotides which hybridise under conditions of high stringency to the described A4 polynucleotides. As used herein conditions of high stringency means hybridisation will occur only if there is at least 90% and preferably 95% identity between the sequences. In a preferred embodiment, the polynucleotides which hybridise to the A4 encoding polynucleotides either retain substantially the same biological function or activity as A4 i.e function as a G protein coupled receptor, or retain the ability to bind the ligand for the receptor even though the polypeptide does not function as a G protein coupled receptor, for example the soluble form of the receptor.

For use in assessing interaction between the receptor and a candidate ligand, it is desirable to construct by application of genetic engineering techniques a mammalian cell that produces an A4 receptor in functional form as a heterologous product or to select a cell line using appropriate screening methods which cell line contains the endogenous nucleic acid sequence for the A4 receptor and expresses such endogenous A4 receptor.

The construction of cell lines is achieved by introducing into a selected host cell a recombinant DNA construct in which DNA coding for the A4 receptor is associated with expression controlling elements that are functional in the selected host to drive expression of the receptor-encoding DNA, and thus elaborate the desired A4 receptor protein. Such cells are herein characterised as having the receptor-encoding DNA incorporated “expressibly” therein. The receptor-encoding DNA is referred to as “heterologous” with respect to the particular cellular host if such DNA is not naturally found in the particular host.

The particular cell type selected to serve as host for production of the A4 receptor can be any of several cell types currently available in the art, including both prokaryotic and eukaryotic, but desirably is not a cell type that in its natural state elaborates a surface receptor that binds an A4 ligand, or analogues thereof, so as to confuse the assay results sought from the engineered cell line. Generally, such problems are avoided by selecting as host cell type which does not express significant levels ofA4, for example,kidney, liver, lung and placenta. Such problems can further be avoided by selecting a non-mammalian cell as a starting material for the analysis. However, it will be appreciated that mammalian cells may nevertheless serve as expression hosts, provided that “background” binding to the test ligand is accounted for in the assay results.

In the alternative, the A4 sequence information herein disclosed allows for the identification of cells expressing endogenous A4receptor, and hence allows for their selection and use in compound screening programs. The use of such A4 receptor producing cells in a screening program is also within the scope of the invention.

According to one embodiment of the present invention, the cell line selected to serve as host for A4 receptor production is a mammalian cell. Several types of such cell lines are currently available for genetic engineering work, and these include the Chinese hamster ovary (CHO) cells for example of K1 lineage (ATCC CCL 61) including the Pro5 variant (ATCC CRL 1281); the fibroblast-like cells derived from SV40-transformed African Green monkey kidney of the CV-1 lineage (ATCC CCL 70), of the COS-1 lineage (ATCC CRL 1650) and of the COS-7 lineage (ATCC CRL 1651); murine L-cells, murine 3T3 cells (ATCC CRL 1658), murine C127 cells, human embryonic kidney cells of the 293 lineage (ATCC CRL 1573), human carcinoma cells including those of the HeLa lineage (ATCC CCL 2), and neuroblastoma cells of the lines IMR-32 (ATCC CCL 127), SK-N-MC (ATCC HTB 10) and SK-N-SH (ATCC HTB 11).

A variety of gene expression systems have been adapted for use with these hosts and are now commercially available, and any one of these systems can be selected to drive expression of the A4 receptor-encoding DNA. These systems, available typically in the form of plasmidic vectors, incorporate expression cassettes the functional components of which include DNA constituting expression controlling sequences, which are host-recognized and enable expression of the receptor-encoding DNA when linked 5′ thereof. The systems further incorporate DNA sequences which terminate expression when linked 3′ of the receptor-encoding region. Thus, for expression in the selected mammalian cell host, there is generated a recombinant DNA expression construct in which DNA coding for the receptor is linked with expression controlling DNA sequences recognized by the host, and which include a region 5′ of the receptor-encoding DNA to drive expression, and a 3′ region to terminate expression. The plasmidic vector harbouring the expression construct typically incorporates such other functional components as an origin of replication, usually virally-derived, to permit replication of the plasmid in the expression host and desirably also for plasmid amplification in a bacterial host, such as E.coli. To provide a marker enabling selection of stable transformed recombinant cells, the vector will also incorporate a gene conferring some survival advantage on the transformants, such as a gene coding for G418 resistance in which case the transformants are plated in medium supplemented with G418.

Included among the various recombinant DNA expression systems that can be used to achieve mammalian cell expression of the receptor-encoding DNA are those that exploit promoters of viruses that infect mammalian cells, such as the promoter from the cytomegalovirus (CMV), the Rous sarcoma virus (RSV), simian virus (SV40), murine mammary tumour virus (MMTV) and others. Also useful to drive expression are promoters such as the LTR of retroviruses, insect cell promoters such as those regulated by temperature, and isolated from Drosophila, as well as mammalian gene promoters such as those regulated by heavy metals, i.e. the metallothionein gene promoter, and other steroid-inducible promoters.

For incorporation into the recombinant DNA expression vector, DNA coding for the desired A4 receptor, can be obtained by applying selected techniques of gene isolation or gene synthesis. The human A4 receptor is expressed in human adult kidney, liver, lung and placenta tissue, and can therefore be obtained by careful application of conventional gene isolation and cloning techniques. This typically will entail extraction of total messenger RNA from a fresh source of human adult kidney, liver, lung and placenta tissue followed by conversion of messenger RNA to cDNA and formation of a library in for example a bacterial plasmid, more typically a bacteriophage. Such bacteriophage harbouring fragments of the human DNA are typically grown by plating on a lawn of susceptible E. coli bacteria, such that individual phage plaques or colonies can be isolated. The DNA carried by the phage colony is then typically immobilized on a nitrocellulose or nylon-based hybridisation membrane, and then hybridized, under carefully controlled conditions, to a radioactively (or otherwise) labelled oligonucleotide probe of appropriate sequence to identify the particular phage colony carrying receptor-encoding DNA or fragment thereof. Typically, the gene or a portion thereof so identified is subcloned into a plasmidic vector for nucleic acid sequence analysis.

An acceptable alternative to using the hybridisation screening method described above for isolating the desired A4 DNA is the PCR homology method. This method of PCR is described in detail in the examples herein. Generally this method involves the amplification of DNA containing specific sequences which are selected via hybridisation to specific primer sequences.

In a specific embodiment of the invention, the A4 receptor is encoded by the DNA sequence illustrated in FIG. 1. In obvious alternatives, the DNA sequence of FIG. 1 may be modified to incorporate synonymous codon equivalents while maintaining a DNA sequence that encodes the A4 receptor.

Having herein provided the nucleotide sequence of a human A4 receptor, it will be appreciated that automated techniques of gene synthesis and/or amplification can be performed to generate DNA coding therefor. Because of the length of A4 receptor-encoding DNA, application of automated synthesis may require staged gene construction, in which regions of the gene up to about 300 nucleotides in length are synthesised individually and then ligated in correct succession for final assembly. Individually synthesised gene regions can be amplified prior to assembly, using polymerase chain reaction (PCR) technology.

The application of automated gene synthesis techniques provides an opportunity for generating sequence variants. It will be appreciated, for example and as mentioned above, that polynucleotides coding for the A4receptor herein described can be generated by substituting synonymous codons for those represented in the polynucleotide sequence herein identified. In addition, polynucleotides coding for synthetic variants of the A4 receptor herein described can be generated which incorporate one or more single amino acid substitutions, deletions or additions. Since it will for the most part be desirable to retain the natural ligand binding profile of the A4 receptor for screening purposes, it is desirable to limit amino acid substitutions to the so-called conservative replacements in which amino acids of like charge are substituted, and to limit substitutions to those sites less critical for receptor activity.

Alternatively, with appropriate template DNA in hand, the technique of PCR amplification may also be used to directly generate all or part of the final gene. In this case, primers are synthesized which will prime the PCR amplification of the final product, either in one piece, or in several pieces that may be ligated together. This may be via step-wise ligation of blunt-ended, amplified DNA fragments, or preferentially via step-wise ligation of fragments containing naturally occurring restriction endonuclease sites. In this application, it is possible to use either cDNA or genomic DNA as the template for the PCR amplification. The cDNA template can be obtained from commercially available or self-constructed cDNA libraries. Specifically, the cDNA template for the A4 receptor can be obtained from the I.M.A.G.E. Consortium with accession number AA449919, submitted to GenBank on Jun. 4, 1997.

Once obtained, the receptor-encoding DNA is incorporated for expression into any suitable expression vector, and host cells are transfected therewith using conventional procedures, such as DNA-mediated transformation, electroporation, microinjection, or particle gun transformation. Expression vectors may be selected to provide transformed cell lines that express the receptor-encoding DNA either transiently or in a stable manner. For transient expression, host cells are typically transformed with an expression vector harbouring an origin of replication functional in a mammalian cell. For stable expression, such replication origins are unnecessary, but the vectors will typically harbour a gene coding for a product that confers on the transformants a survival advantage, to enable their selection. Genes coding for such selectable markers include the E. coli gpt gene which confers resistance to mycophenolic acid, the neo gene from transposon Tn5 which confers resistance to the antibiotic G418 and to neomycin, the dhfr sequence from murine cells or E. coli which changes the phenotype of DHFR− cells into DHFR+ cells, and the tk gene of herpes simplex virus, which makes TK− cells phenotypically TK+ cells. Both transient expression and stable expression can provide transformed cell lines, and membrane preparations derived therefrom, for use in ligand screening assays.

For use in screening assays, cells transiently expressing the receptor-encoding DNA can be stored frozen for later use, but because the rapid rate of plasmid replication will lead ultimately to cell death, usually in a few days, the transformed cells should be used as soon as possible. Such assays may be performed either with intact cells, or with membrane preparations derived from such cells. The membrane preparations typically provide a more convenient substrate for the ligand binding experiments, and are therefore preferred as binding substrates. To prepare membrane preparations for screening purposes, i.e., ligand binding experiments, frozen intact cells are homogenized while in cold binding buffer suspension and a membrane pellet is collected after centrifugation. The membranes may then be used as such, or after storage in lyophilized form, in the ligand binding assays. Alternatively, intact, fresh cells harvested about two days after transient transfection or after about the same period following fresh plating of stable transfected cells, can be used for ligand binding assays by the same methods as used for membrane preparations. When cells are used, the cells must be harvested by more gentle centrifugation so as not to damage them, and all washing must be done in a buffered medium, for example in phosphate-buffered saline, to avoid osmotic shock and rupture of the cells.

In an alternative to using cells that express receptor-encoding DNA, ligand characterization may also be performed using cells, for example Xenopus oocytes, that yield functional membrane-bound receptor following introduction of messenger RNA coding for the A4 receptor. In this case, the A4 receptor gene of the invention is typically subcloned into a plasmidic vector such that the introduced gene may be easily transcribed into RNA via an adjacent RNA transcription promoter supplied by the plasmidic vector, for example the T3 or T7 bacteriophage promoters. RNA is then transcribed from the inserted gene in vitro, and can then be injected into Xenopus oocytes. Following the injection of an RNA solution, the oocytes are left to incubate for up to several days, and are then tested in either intact or membrane preparations form for the ability to bind a particular ligand molecule supplied in a bathing solution.

The interaction of a candidate ligand with a selected A4 receptor of the invention is evaluated typically by determining receptor/ligand binding. In one embodiment, the interaction of ligands with an A4 receptor of the present invention can be determined by measuring a functional receptor/ligand interaction such as an electrophysiological interaction, by screening test ligands for their ability to modulate ion channel activity. The present invention thus further provides, as a ligand screening technique, a method of detecting interaction between a test ligand and an A4 receptor, which comprises the steps of incubating the test ligand with a A4 receptor-producing cell or with a membrane preparation derived therefrom, and then measuring ligand-induced electrical current across said cell or membrane using microelectrodes inserted into the cell or placed on either side of a cell-derived membrane preparation using the “patch-clamp” technique or a microphysiometer.

The interaction of a ligand with a A4 receptor can also be determined by assaying second messenger response associated with the A4 receptor activity to determine the ability of a given ligand to modulate A4 receptor activity. Furthermore, such second messenger response provides a means to differentiate antagonistic ligands from agonistic ligands. Such second messengers include, for example, cyclic AMP (cAMP) and intracellular calcium ion (Ca++). Thus, depending on the nature of the interaction, i.e. stimulatory or inhibitory, an increase or a decrease in intracellular cAMP or Ca++ can be measured to determine the extent of receptor/ligand interaction, using established assays. In a preferred embodiment, an A4 receptor-expressing cells in accordance with the present invention is subjected to adenylyl cyclase stimulant treatment, e.g. with forskolin, followed by incubation with a candidate ligand and a labelled substrate for adenylyl cyclase, e.g. [.32P]ATP, and then determining the extent of ligand-induced adenylyl cyclase activity, e.g. by determining the conversion of [.32P]ATP to [32P]cAMP. Techniques such as those described in Salomon et al. in Anal. Biochem., 1974, 58:541 are useful to determine the conversion of ATP to cAMP.

In addition to using the receptor-encoding DNA to construct cell lines useful for ligand screening, expression of the DNA can, according to another aspect of the invention, be performed to produce fragments of the receptor in soluble form, for structure investigation, to raise antibodies and for other experimental uses. It will be appreciated that the production of such fragments may be accomplished in a variety of host cells. Mammalian cells such as CHO cells may be used for this purpose, the expression typically being driven by an expression promoter capable of high-level expression, for example the CMV (cytomegalovirus) promoter. Alternately, non-mammalian cells, such as insect Sf9 (Spodoptera frugiperda) cells may be used, with the expression typically being driven by expression promoters of the baculovirus, for example the strong, late polyhedrin protein promoter. Filamentous fungal expression systems may also be used to secrete large quantities of such domains of the A4receptor. Aspergillus nidulans, for example, with the expression being driven by the alcA promoter, would constitute such an acceptable system. In addition to such expression hosts, it will be further appreciated that any prokaryotic or other eukaryotic expression system capable of expressing heterologous genes or gene fragments, whether intracellularly or extracellularly would be similarly acceptable.

For use particularly in detecting the presence and/or location of an A4receptor, for example in kidney, liver, lung and placenta tissue, the present invention also provides, in another of its aspects, labelled antibody to a human A4receptor. To raise such antibodies, there may be used as immunogen either the intact, soluble receptor or an immunogenic fragment thereof, produced in a microbial or mammalian cell host as described above or by standard peptide synthesis techniques. Regions of the A4 receptor particularly suitable for use as immunogenic fragments include those corresponding in sequence to an extracellular region of the receptor, or a portion of the extracellular region, such as peptides consisting of residues 1-45, and peptides corresponding to the region between transmembrane domains TM-2 and TM-3, such as a peptide consisting of residues 105-120, between transmembrane domains TM-4 and TM-5, such as a peptide consisting of residues 183-217 and between transmembrane domains TM-6 and TM-7, such as a peptide consisting of residues 298-311. Peptides derived from intracellular loop domains are also appropriate for use in raising antibodies such as peptides corresponding to the region between transmembrane domains TM-1 and TM-2, such as residues 70-81, the region between transmembrane domains TM-3 and TM-4, such as residues 142-159, and the region between transmembrane domains TM-5 and TM-6, such as residues 241-274. Peptides consisting of the C-terminal domain 337-420, or fragments thereof may also be used for the raising of antibodies.

The raising of antibodies to the desired A4 receptor or immunogenic fragment can be achieved, for polyclonal antibody production, using immunization protocols of conventional design, and any of a variety of mammalian hosts, such as sheep, goats and rabbits. Alternatively, for monoclonal antibody production, immunocytes such as splenocytes can be recovered from the immunized animal and fused, using hybridoma technology, to myeloma cells. The fusion products are then screened by culturing in a selection medium, and cells producing antibody are recovered for continuous growth, and antibody recovery. Recovered antibody can then be coupled covalently to a detectable label, such as a radiolabel, enzyme label, luminescent label or the like, using linker technology established for this purpose.

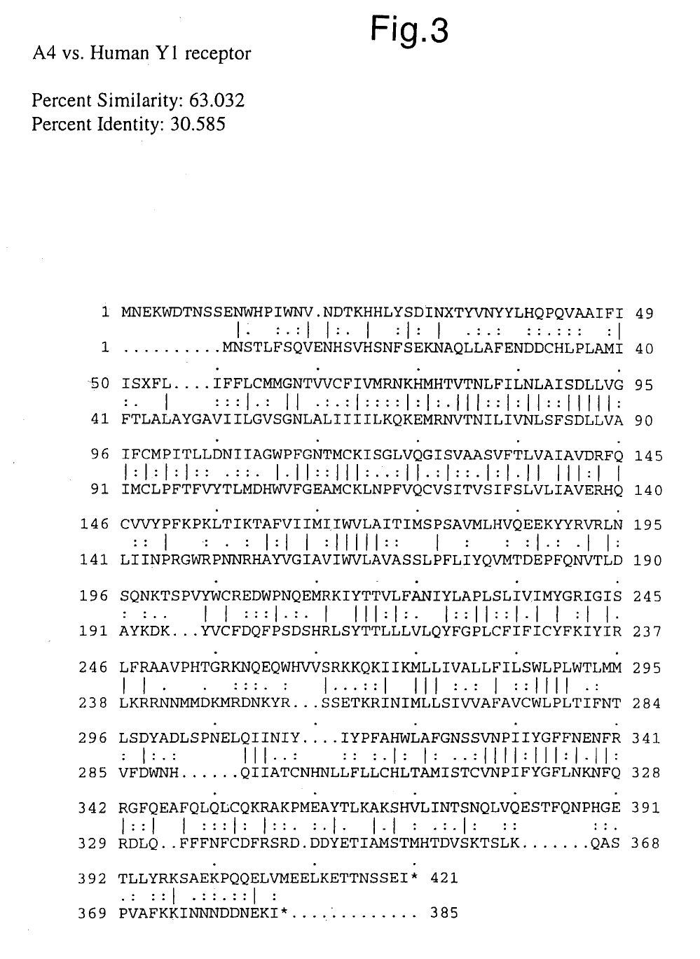

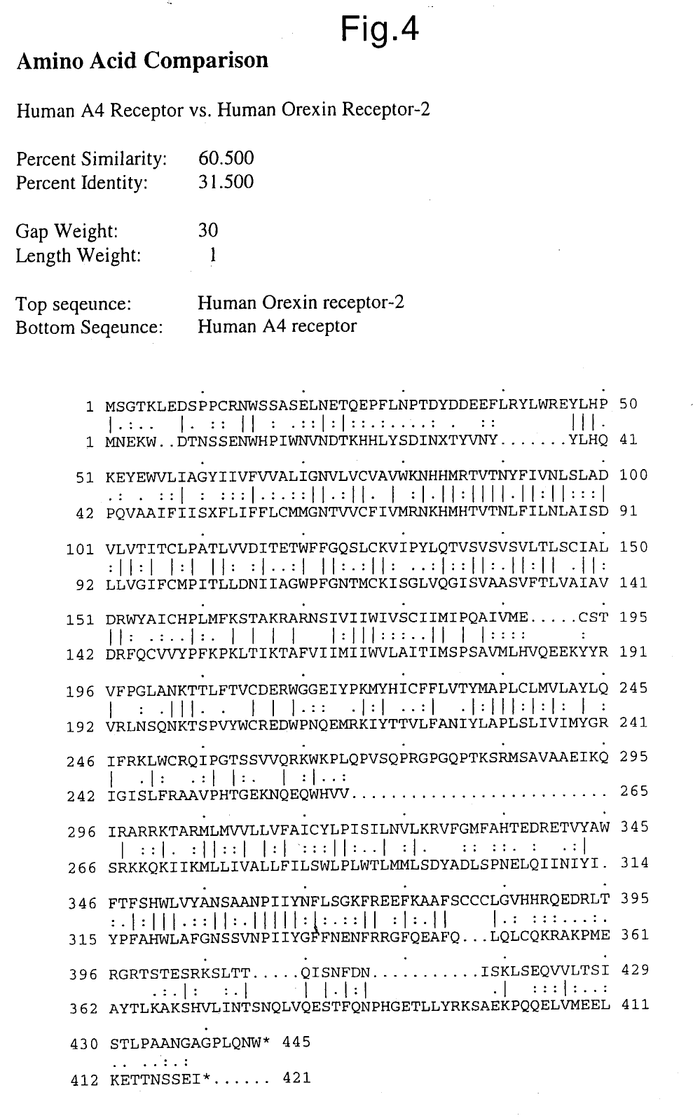

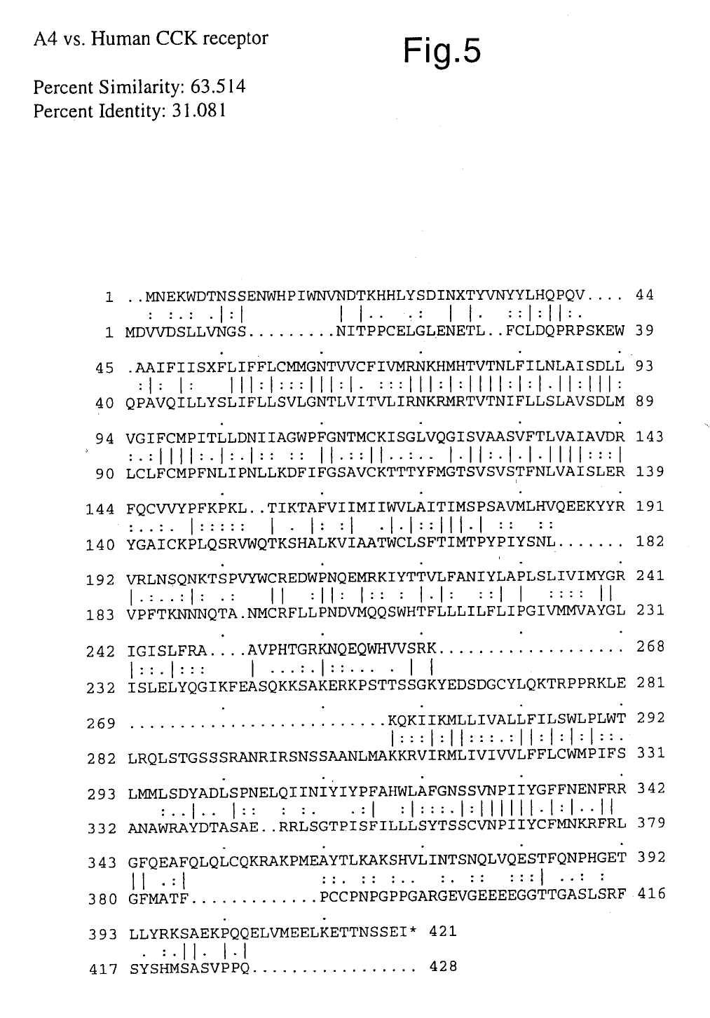

In detectably labelled form, e.g. radiolabelled form or non-radiolabelled forms such as chemiluminescent forms, DNA or RNA coding for human A4 receptors, and selected regions thereof, may also be used, in accordance with another aspect of the present invention, as hybridisation probes for example to identify sequence-related genes resident in the human or other mammalian genomes (or cDNA libraries) or to locate A4-encoding DNA in a specimen, such as kidney, liver, lung and placenta tissue. This can be done using either the intact coding region, or a fragment thereof having radiolabelled nucleotides, e.g. 32P, incorporated therein. To identify the A4-encoding DNA in a specimen, it is desirable to use either the full length cDNA coding therefor, or a fragment which is unique thereto: preferably, such fragments are at least 15 nucleotides long. These unique regions can be identified by aligning the human A4 nucleotide sequences provided herein with the nucleotide sequences of the most closely related known G protein coupled receptors. (See FIGS. 3-5).

Embodiments of the invention are described in the following specific examples which are not to be construed as limiting.

### EXAMPLE 1

### PCR Cloning of the Full Length A4 cDNA Clone

An I.M.A.G.E. Consortium EST clone with accession number AA449919, submitted to GenBank on Jun. 4, 1997, showed homology to known NPY receptors. The clone was isolated from cDNA library prepared from mRNA obtained from pooled 8-9 week human (total) fetus material. This cDNA clone was purchased (Research Genetic; Cat. No. 97002) and sequenced by the dideoxy chain termination method on an Applied Biosystems Model 377 fluorescent dye DNA sequencer. The predicted polypeptide encoded by this cDNA clone showed high homology to the carboxy end of NPY receptors but lacked approximately 25% on amino-terminal region of the receptor including the initiation methionine.

To identify sequences corresponding to the 5′-end of AA449919 open reading frame a 5′-RACE PCR technique was utilized. Two 5′ directed primers, sequence P1 (5′ GAGACATAATGGTGATGGCTAGGACCCA 3′) and P2 (5′ CTGCGACAGATATTCCCTGGACCAATCC 3′) were designed based on the sequence of the AA449919 cDNA clone. These oligonucleotide primers were used in a 5′RACE PCR procedure to obtain the upstream sequences from human brain Marathon-Ready™ cDNA Amplification Kit (Clontech Laboratories Inc.; Cat. No. 7400-1) according to the manufacturers recommendations. Human brain cDNA was amplified using primer P1 and the adaptor primer AP1, (5′ CCATCCTAATACGACTCACTATAGGC 3′; Clontech) under the following PCR conditions: 1 min at 94° C.; 5 cycles of 30 seconds at 94° C. then 4 minutes at 72° C.; 5 cycles of at 94° C. then 4 minutes at 70° C.; 25 cycles of 30 seconds at 94° C. then 4 minutes at 68°C.; 10 minutes at 68° C. An aliquot of this PCR reaction was diluted and re-amplified under the same cycling conditions using the primer AP2 (5′ACTCACTATAGGGCTCGAGCGGC 3′) which is nested with respect to AP1, and primer P2, which is nested with respect to P1. An aliquot of this reaction was electrophoresed on a 1% agarose gel. A band of 600 bp was visible by ethidium bromide staining. Eluate of this band was re-amplified with primers AP2 and P2 under the following PCR conditions: 1 min at 94° C.; 30 cycles of 30 seconds at 94° C., 30 seconds at 70° C., 1 minute at 72° C.; 10 minutes at 72° C. An aliquot of this PCR reaction was run on a gel and ethidium bromide staining revealed a band at the expected size of 600 bp. An aliquot of the PCR reaction was used directly for ligation to the vector pCR 2.1 (Invitrogen, Cat. No. K2030) and transformed into Top 10F′ bacterial cells. The resulting clones were sequenced by the dideoxy chain termination method on an Applied Biosystems Model 377 fluorescent dye DNA sequencer. This 600 bp clone overlapped the AA449919 cDNA sequence and included sequences representing the entire 5′ end of the open reading frame including the codon representing the initiating methionine as well as some 5′ UTR sequences.

### EXAMPLE 2

### Reconstruction of a Full-Length Human A4 Clone Using PCR

The DNA sequence encoding for the novel receptor A4 was amplified using oligonucleotide primers corresponding to the 5′ and 3′ end of the cDNA. The 5′ oligonucleotide primer, termed PA4-5, has the sequence 5′-GGCATTCGAATTCGCCGCCACCATGAATGAGAAATGGGACACAAACTCTT-3′ and contains a EcoRI restriction site and a consensus Kozak translation initiation sequence followed by 28 nuccleotides of the AA449919 sequence starting from the methionine start codon. The 3′ oligonucleotide primer, termed PA4-3, has the sequence site (5′-AGGATTATCACTCTAGATCTTTTTAAATCTCACTGCTGTTAGTAGTTTCT-3′and contains 33 bases of the 3′ UTR of the AA449919 sequence and a XbaI restriction site. A full-length sequence encoding for the A4 receptor was obtained using a two step procedure. First, two cDNA clones corresponding to the 5′ and 3′ ends of the AA449919 open reading frame were amplified in seperate reactions. Second, the two products were combined and reamplified in the presence of human kidney Marathon-Ready™ cDNA with appropriate primers to give a full-length A4 cDNA clone. Briefly, amplification of the 5′ fragment was performed using the human kidney Marathon-Ready™ cDNA Amplification Kit (Clontech Laboratories Inc.; Cat. No. 7405-1) with primers PA4-5 and P1 under the following PCR conditions: 1 min at 94° C.; 25 cycles of 30 seconds at 94° C., 4 minutes at 72° C.; 10 minutes at 72° C. An aliquot of this reaction was reamplified under the same conditions and produced a band of the proper size when an aliquot of the reaction was run on an ethidium bromide stained agarose gel. Amplification of the 3′ fragment was also performed using human kidney cDNA by amplification with primer P5 (5′-TGGCACGTGGTGTCCAGGAAGAAGCAG-3′) and PA4-3 under the following PCR conditions: 1 min at 94° C.; 35 cycles of 30 seconds at 94° C., 30 seconds at 65° C., 4 minutes at 72° C. A strong band of the proper size was visible when an aliquot of the reaction was run on an ethidium bromide stained agarose gel. Next, to produce a full-length cDNA human kidney marathon cDNA was combined with the two above PCR products and extended using PA4-5 and PA4-3 primers under the following conditions: 1 min at 94° C.; 35 cycles of 30 seconds at 94° C., 30 seconds at 65° C., 4 minutes at 72° C.; 10 minutes at 72° C. An aliquot of the first round of PCR was then re-amplified under identical conditions except the cycle number was increased to 35. A strong band of the proper size(1.4 kilobases) was visible when an aliquot of the reaction was run on an ethidium bromide stained agarose gel. An aliquot of the PCR reaction was restriction digested with the enzymes EcoRI and HindIII and electrophoresed on an 1% agarose gel. The PCR product was excised, purified, ligated into the EcoRI/XbaI sites of the mammlian expression vector pcDNA3 (Invitrogen). The resulting construct, named pcDNA3-A4. Orientation of the cDNA was confirmed by restriction digestion analysis and sequencing.

### EXAMPLE 3

### Tissue Distribution of Human A4 Receptor mRNA

Tissue distribution of A4 was determined by probing a Human RNA Master Blot (Clontech, Cat No. 7770-1) using a radiolabeled full-length A4 cDNA as probe according to the manufacturers recommendations. Briefly, the blot was prehybridized in ExpressHyb™ Hybridization Solution (Clontech, Cat. No. 8015-1) overnight at 65° C. Next, hybridization was performed overnight at 65° C. in fresh ExpressHyb™ with ³²P-labelled AA449919 cDNA (3×10⁶ cpm/mL). The filters were washed to a stringency of 2×SSPE/0.1% SDS at 65° C. and exposed for three days onto Kodak X-OMAT film. Positive signals were observed in adult kidney, liver, lung and placenta.

### EXAMPLE 4

### Chromosomal Localization

The procedure for FISH detection was performed to determine the chromosomal localisation of the A4 receptor.

(a) Slides Preparation

Lymphocytes isolated from human blood were cultured in α-minimal essential medium (MEM) supplemented wiwth 10% fetal calf serum and phytohemagglutinin (PHA) at 37° C. for 68-72 hr. The lymphocyte cultures were treated with BrdU (0.1 8 mg/ml Sigma) to synchronize the cell population. The synchronized cells were washed three times with serum free medium to release the block and recultured at 37° C. for 6 hr in a MEM with thymidine (2.5 μg/ml: Sigma). Cells were harvested and slides were made by using standard procedures including hypotonic treatment, fix and air-dry.

(b) In Situ Hybridization and FISH Detection

BAC probe was biotinylated with dATP using the BRL BioNick labelling kit (15° C., 2 hr) (Heng et al, High Resolution Mapping of Mammalian Genes by in situ Hybridization to Free Chromatin. Proc. NatI Aca Sci USA 89: 9509-9513, 1992)

The procedure for FISH detection was performed according to Heng et al., 1992 and Heng and Tsui 1993 (Modes of DAPI banding and simultaneous in situ hybrization. Chromosoma. 102: 325-332 (1993)). Briefly, slides were baked at 55° C. for 1 hr. After RNase treatment, the slides were denatured in 70% formamide in 2×SSC for 2 min. in 70° C. followed by dehydration with ethanol. Probes were denatured at 75° C. for 5 min. in a hybridization mix consisting of 50% formamide and 10% dextran sulphate. Probes were loaded on the denatured chromosomal slides. After overnight hybridisation, slides were washed and detected as well as amplified. FISH signals and the DAPI banding pattern was recorded separately by taking photographs, and the assignment of the FISH mapping data with chromosomal bands was achieved by superimposing FISH signals with DAPI banded chromosomes (Heng and Tsui, 1993).

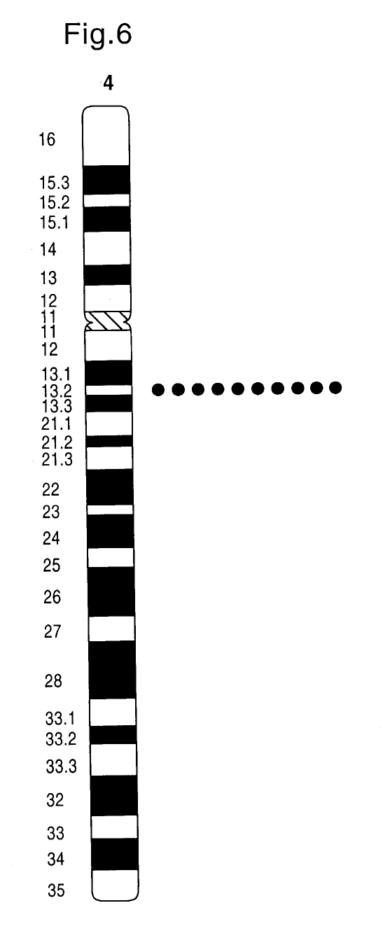

Two regions of one chromosome showed the FISH positive. Under the conditions used, the hybridisation efficiency was approximately 98% for this probe (among 100 checked mitotic figures, 98 of them showed signals on one pair of the chromosomes). Since the DAPI banding was used to identify the specific chromosome, the assignment between signal from probe and the long arm of chromosome 4 was obtained. The detailed position was further determined based on the summary from 10 photographs as set out in FIG. 6.

### EXAMPLE 5

### Antisense Analysis

Knowledge of the correct, complete cDNA sequence of A4 enables its use as a tool for antisense technology in the investigation of gene function. Oligonucleotides, cDNA or genomic fragments comprising the antisense strand of A4 are used either in vitro or in vivo to inhibit expression of the mRNA. Such technology is now well known in the art, and antisense molecules can be designed at various locations along the nucleotide sequences. By treatment of cells or whole test animals with such antisense sequences, the gene of interest is effectively turned off. Frequently, the function of the gene is ascertained by observing behavior at the intracellular, cellular, tissue or organism level (e.g., lethality, loss of differentiated function, changes in morphology, etc.).

In addition to using sequences constructed to interrupt transcription of a particular open reading frame, modifications of gene expression is obtained by designing antisense sequences to intron regions, promoter/enhancer elements, or even to trans-acting regulatory genes. Similarly, inhibition is achieved using Hogeboom base-pairing methodology, also known as “triple helix” base pairing.

### EXAMPLE 6

### Testing of Chimeric Seven Transmembrane G Protein Coupled Receptors

Functional chimeric seven transmembrane G protein coupled receptors (GPCRs) are constructed by combining the extracellular and/or transmembrane ligand-receptive sequences of a new isoform with the transmembrane and/or intracellular segments of a different T7G for test purposes. This concept was demonstrated by Kobilka et al (1988, Science 240:1310-1316) who created a series of chimeric α2-β2 adrenergic receptors (AR) by inserting progressively greater amounts of α2-AR transmembrane sequence into β2-AR. The binding activity of known agonists changed as the molecule shifted from having more α2 than β2 conformation, and intermediate constructs demonstrated mixed specificity. The specificity for binding antagonists, however, correlated with the source of the domain VII. The importance of domain VII for ligand recognition was also found in chimeras utilizing two yeast α-factor receptors and is significant because the yeast receptors are classified as miscellaneous receptors. Thus, functional role of specific domains appears to be preserved throughout the GPCR family regardless of category.

In parallel fashion, internal segments or cytoplasmic domains from a particular isoform are exchanged with the analogous domains of a known GPCR and used to identify the structural determinants responsible for coupling the receptors to trimeric G-proteins (Dohiman et al (1991) Annu Rev Biochem 60:653-88). A chimeric receptor in which domains V, VI, and the intracellular connecting loop from β2-AR were substituted into a2-AR was shown to bind ligands with a2-AR specificity, but to stimulate adenylate cyclase in the manner of β2-AR. This demonstrates that for adrenergic-type receptors, G-protein recognition is present in domains V and VI and their connecting loop. The opposite situation was predicted and observed for a chimera in which the V→VI loop from α1-AR replaced the corresponding domain on β2-AR and the resulting receptor bound ligands with β2-AR specificity and activated G-protein-mediated phosphatidylinositol turnover in the α1-AR manner. Finally, chimeras constructed from muscarinic receptors also demonstrated that V→VI loop is the major determinant for specificity of G-protein activity.

Chimeric or modified T7Gs containing substitutions in the extracellular and transmembrane regions have shown that these portions of the receptor determine ligand binding specificity. For example, two Ser residues conserved in domain V of all adrenergic and D catecholamine T7G receptors are necessary for potent agonist activity. These serines are believed to form hydrogen bonds with the catechol moiety of the agonists within the GPCR binding site. Similarly, an Asp residue present in domain III of all GPCRs which bind biogenic amines is believed to form an ion pair with the ligand amine group in the GPCR binding site.

Functional, cloned GPCRs are expressed in heterologous expression systems and their biological activity assessed (e.g. Marullo et al (1988) Proc Natl Acad Sci 85:7551-55; King et al (1990) Science 250:121-23). One heterologous system introduces genes for a mammalian T7G and a mammalian G-protein into yeast cells. The GPCR is shown to have appropriate ligand specificity and affinity and trigger appropriate biological activation, growth arrest and morphological changes, of the yeast cells.

An alternate procedure for testing chimeric receptors is based on the procedure utilizing the P2u purinergic receptor (P2u) as published by Erb et al (1993, Proc Natl Acad Sci 90:104411-53). Function is easily tested in cultured K562 human leukemia cells because these cells lack P2u receptors. K562 cells are transfected with expression vectors containing either normal or chimeric P2u and loaded with fura-a, fluorescent probe for Ca++. Activation of properly assembled and functional P2u receptors with extracellular UTP or ATP mobilizes intracellular Ca++ which reacts with fura-a and is measured spectrofluorometrically. As with the GPCRs above, chimeric genes are created by combining sequences for extracellular receptive segments of any newly discovered GPCR polypeptide with the nucleotides for the transmembrane and intracellular segments of the known P2u molecule. Bathing the transfected K562 cells in microwells containing appropriate ligands triggers binding and fluorescent activity defining effectors of the GPCR molecule. Once ligand and function are established, the P2u system is useful for defining antagonists or inhibitors which block binding and prevent such fluorescent reactions.

### EXAMPLE 7

### Diagnostic Test Using A4 Specific Antibodies

A4 antibodies are useful for investigating signal transduction and the diagnosis of infectious or hereditary conditions which are characterized by differences in the amount or distribution of A4 or downstream products of an active signaling cascade.

Diagnostic tests for A4 include methods utilizing antibody and a label to detect A4 in human body fluids, membranes, cells, tissues or extracts of such. The polypeptides and antibodies of the present invention are used with or without modification. Frequently, the polypeptides and antibodies are labeled by joining them, either covalently or noncovalently, with a substance which provides for a detectable signal. A wide variety of labels and conjugation techniques are known and have been reported extensively in both the scientific and patent literature. Suitable labels include radionuclides, enzymes, substrates, cofactors, inhibitors, fluorescent agents, chemiluminescent agents, chromogenic agents, magnetic particles and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced as shown in U.S. Pat. No. 4,816,567, incorporated herein by reference.

A variety of protocols for measuring soluble or membrane-bound A4, using either poyclonal or monoclonal antibodies specific for the protein, are known in the art. Examples include enzyme-linked immunosorbent assay (ELISA), radioimmunoassay (RIA) and fluorescent activated cell sorting (FACS). A two-site monoclonal-based immunoassay utilizing monoclonal antibodies reactive to two non-interfering epitopes on A4 is preferred, but a competitive binding assay may be employed. These assays are described, among other places, in Maddox, DE et al (1983, J Exp. Med. 158:121 1f).

### EXAMPLE 8

### Purification of Native A4 Using Specific Antibodies

Native or recombinant A4 is purified by immunoaffinity chromatography using antibodies specific for A4. In general, an immunoaffinity column is constructed by covalently coupling the anti-TRH antibody to an activated chromatographic resin.

Polyclonal immunoglobulins are prepared from immune sera either by precipitation with ammonium sulfate or by purification on immobilized Protein A (Pharmacia LKB Biotechnology, Piscataway N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated Sepharose (Pharmacia LKB Biotechnology). The antibody is coupled to the resin, the resin is blocked, and the derivative resin is washed according to the manufacturer's instructions.

Such immunoaffinity columns are utilized in the purification of A4 by preparing a fraction from cells containing A4 in a soluble form. This preparation is derived by solubilization of whole cells or of a subcellular fraction obtained via differential centrifugation (with or without addition of detergent) or by other methods well known in the art. Alternatively, soluble A4 is secreted in useful quantity into the medium in which the cells are grown.

A soluble A4-containing preparation is passed over the immunoaffinity column, and the column is washed under conditions that allow the preferential absorbance of A4 (e.g., high ionic strength buffers in the presence of detergent). Then, the column is eluted under conditions that disrupt antibody/protein binding (e.g., a buffer of pH 2-3 or a high concentration of a chaotrope such as urea or thiocyanate ion), and A4 is collected.

### EXAMPLE 9

### Drug Screening

This invention is particularly useful for screening therapeutic compounds by using A4 or binding fragments thereof in any of a variety of drug screening techniques. As A4 is a G protein coupled receptor any of the methods commonly used in the art may potentially used to identify A4 ligands. For example, the activity of a G protein coupled receptor such as A4 can be measured using any of a variety of appropriate functional assays in which activation of the receptor results in an observable change in the level of some second messenger system, such as adenylate cyclase, guanylyl cyclase, calcium mobilization, or inositol phospholipid hydrolysis. One such approach, measures the effect of ligand binding on the activation of intracellular second messenger pathways, using a reporter gene. Typically, the reporter gene will have a promoter which is sensitive to the level of that second messenger controlling expression of an easily detectable gene product, for example, CAT or luciferase. Alternatively, the cell is loaded with a reporter substance, e.g., FURAwhereby changes in the intracellular concentration of calcium indicate modulation of the receptor as a result of ligand binding. Thus, the present invention provides methods of screening for drugs or any other agents which affect signal transduction.

Alternatively, the polypeptide or fragment employed in such a test is either free in solution, affixed to a solid support, borne on a cell surface or located intracellularly. One method of drug screening utilizes eukaryotic or prokaryotic host cells which are stable transformed recombinant nucleic acids expressing the polypeptide or fragment. Drug candidates are screened against such transformed cells in competitive binding assays. Such cells, either in viable or fixed form, are used for standard binding assays. One measures, for example, the formation of complexes between A4 and the agent being tested. Alternatively, one examines the diminution in complex formation between A4 and a ligand caused by the agent being tested.

This invention also contemplates the use of competitive drug screening assays in which neutralizing antibodies capable of binding A4 specifically compete with a test compound for binding to A4 polypeptides or fragments thereof. In this manner, the antibodies are used to detect the presence of any peptide which shares one or more antigenic determinants with A4.

### EXAMPLE 10

### Use and Administration of Antibodies, Inhibitors, or Antagonists

Antibodies, inhibitors, or antagonists of A4 (or other treatments to limit signal transduction, LST) provide different effects when administered therapeutically. LSTs are formulated in a nontoxic, inert, pharmaceutically acceptable aqueous carrier medium preferably at a pH of about 5 to 8, more preferably 6 to 8, although pH may vary according to the characteristics of the antibody, inhibitor, or antagonist being formulated and the condition to be treated. Characteristics of LSTs include solubility of the molecule, half-life and antigenicity/immunogenicity. These and other characteristics aid in defining an effective carrier.

LSTs are delivered by known routes of administration including but not limited to topical creams and gels; transmucosal spray and aerosol; transdermal patch and bandage; injectable, intravenous and lavage formulations; and orally administered liquids and pills particularly formulated to resist stomach acid and enzymes. The particular formulation, exact dosage, and route of administration is determined by the attending physician and varies according to each specific situation.

Such determinations are made by considering multiple variables such as the condition to be treated, the LST to be administered, and the pharmacokinetic profile of a particular LST. Additional factors which are taken into account include severity of the disease state, patient's age, weight, gender and diet, time and frequency of LST administration, possible combination with other drugs, reaction sensitivities, and tolerance/response to therapy. Long acting LST formulations might be administered every 3 to 4 days, every week, or once every two weeks depending on half-life and clearance rate of the particular LST.

Normal dosage amounts vary from 0.1 to 100,000 micrograms, up to a total dose of about 1 g, depending upon the route of administration. Guidance as to particular dosages and methods of delivery is provided in the literature; see U.S. Pat. Nos. 4,657,760; 5,206,344; or 5,225,212. Those skilled in the art employ different formulations for different LSTs. Administration to cells such as nerve cells necessitates delivery in a manner different from that to other cells such as vascular endothelial cells.

It is contemplated that abnormal signal transduction, trauma, or diseases which trigger A4 activity are treatable with LSTs. These conditions or diseases are specifically diagnosed by the tests discussed above, and such testing should be performed in suspected cases of viral, bacterial or fungal infections: allergic responses; mechanical injury associated with trauma; hereditary diseases; lymphoma or carcinoma; or other conditions which activate the genes of lymphoid or neuronal tissues.

### EXAMPLE 11

### Production of Transgenic Animals

Animal model systems which elucidate the physiological and behavioral roles of the A4 receptor are produced by creating transgenic animals in which the activity of the A4 receptor is either increased or decreased, or the amino acid sequence of the expressed A4 receptor is altered, by a variety of techniques. Examples of these techniques include, but are not limited to: 1) Insertion of normal or mutant versions of DNA encoding a A4 receptor, by microinjection, electroporation, retroviral transfection or other means well known to those skilled in the art, into appropriate fertilized embryos in order to produce a transgenic animal or 2) Homologous recombination of mutant or normal, human or animal versions of these genes with the native gene locus in transgenic animals to alter the regulation of expression or the structure of these A4 receptor sequences. The technique of homologous recombination is well known in the art. It replaces the native gene with the inserted gene and so is useful for producing an animal that cannot express native A4 receptors but does express, for example, an inserted mutant A4 receptor, which has replaced the native A4 receptor in the animal's genome by recombination, resulting in underexpression of the transporter. Microinjection adds genes to the genome, but does not remove them, and so is useful for producing an animal which expresses its own and added A4 receptors, resulting in overexpression of the A4 receptor.

One means available for producing a transgenic animal, with a mouse as an example, is as follows: Female mice are mated, and the resulting fertilized eggs are dissected out of their oviducts. The eggs are stored in an appropriate medium such as M2 medium. DNA or cDNA encoding a A4 purified from a vector by methods well known in the art. Inducible promoters may be fused with the coding region of the DNA to provide an experimental means to regulate expression of the transgene. Alternatively or in addition, tissue specific regulatory elements may be fused with the coding region to permit tissue-specific expression of the trans-gene. The DNA, in an appropriately buffered solution, is put into a microinjection needle (which may be made from capillary tubing using a piper puller) and the egg to be injected is put in a depression slide. The needle is inserted into the pronucleus of the egg, and the DNA solution is injected. The injected egg is then transferred into the oviduct of a pseudopregnant mouse (a mouse stimulated by the appropriate hormones to maintain pregnancy but which is not actually pregnant), where it proceeds to the uterus, implants, and develops to term. As noted above, microinjection is not the only methods for inserting DNA into the egg cell, and is used here only for exemplary purposes.

All publications and patents mentioned in the above specification are herein incorporated by reference.

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

We claim:

1. An isolated polynucleotide encoding a receptor wherein said polynucleotide is selected from the group consisting of:
a) a polynucleotide encoding a polypeptide having the deduced amino acid sequence of FIG. 1 or a fragment, analog or derivative of said polypeptide; and
b) a polynucleotide capable of hybridising to and which is at least 70% identical to the polynucleotide of FIG. 1.

2. An isolated polynucleotide according to claim 1, wherein the polynucleotide is the polynucleotide of FIG. 1.

3. An isolated polynucleotide according to claim 1, wherein the polynucleotide encodes a polypeptide having the deduced amino acid sequence of FIG. 1 or a fragment, analog or derivative of said polypeptide.

4. An isolated polynucleotide comprising a region that encodes a variant of the polynucleotide of FIG. 1, said variant sharing at least 95% amino acid identity with said FIG. 1 polynucleotide

5. A recombinant DNA construct having incorporated therein a polynucleotide as defined in any one of claims 1 to 4.

6. A cell that has been selected to produce a receptor encoded by the polynucleotide as defined in any one of claims 1 to 4.

7. A cell according to claim 6 wherein said cell is genetically engineered to produce said receptor by incorporating expressibly therein a recombinant construct as defined in claim 5.

8. A cell according to claim 6 wherein said receptor is expressed endogenously.

9. A cell as defined in claim 6 which is a mammalian cell.

10. A receptor-binding membrane preparation derived from a cell as defined in claim 6.

11. A method of assaying a test ligand for binding with a receptor encoded by the polynucleotide as defined in any one of claims 1 to 4, which comprises the steps of incubating the test ligand under appropriate conditions with a receptor-producing cell as defined in claim 6, or with membrane preparation derived therefrom, and then determining whether binding between said receptor and said test ligand has occured.

12. A method according to claim 11 wherein the binding between said receptor and said test ligand is determined by measuring a functional receptor response.

13. A method as defined in claim 12, wherein said functional receptor response is a second messenger response.

14. A method as defined in claim 13, wherein said second messenger is selected from the group consisting of intracellular cAMP and intracellular calcium ion.

15. A receptor encoded by the polynucleotide as defined in any one of claims 1 to 4, in an isolated form essentially free from other proteins of human origin.

16. A ligand-binding fragment of a receptor encoded by the polynucleotide defined in any one of claims 1 to 4.

17. An antibody which binds a mammalian receptor encoded by the polynucleotide defined in any one of claims 1 to 4.

18. An immunogenic fragment of a human receptor wherein said receptor is encoded by the polynucleotide defined in any one of claims 1 to 4.

19. An oligonucleotide which comprises at least about 17 nucleic acids and which selectively hybridizes with a polynucleotide defined in claim 1 or complement thereof.
